(19) United States
(12) Patent Application Publication
(10) Pub. No.: US 2025/0114209 A1
(43) Pub. Date: Apr. 10, 2025

(54) SYSTEM AND METHOD FOR MINIMALLY-INVASIVE BONE FUSION

(71) Applicant: WRIGHT MEDICAL TECHNOLOGY, INC., Memphis, TN (US)

(72) Inventors: Zachary KORMAN, St. Louis, MO (US); David Jens DALSTROM, Seattle, WA (US)

(73) Assignee: WRIGHT MEDICAL TECHNOLOGY, INC., Memphis, TN (US)

(21) Appl. No.: 18/823,785

(22) Filed: Sep. 4, 2024

Related U.S. Application Data
(60) Provisional application No. 63/588,828, filed on Oct. 9, 2023.

(51) Int. Cl. A61F 2/42; A61B 17/17; A61F 2/30; A61F 2/46

(52) U.S. Cl. CPC A61F 2/4225; A61B 17/1728; A61B 17/1775; A61F 2/30749; A61F 2/4606; A61F 2002/30622; A61F 2002/4233

(57) ABSTRACT

A system for bone fusion incudes an implant configured to be attached to two bone portions; an insertion handle attached to the implant by a threaded drill guide and including a stability screw trajectory guide portion and an implant screw guide portion; and a stability screw configured to pass through the stability screw trajectory guide portion and into the two bones.

### CROSS REFERENCE TO RELATED APPLICATIONS

This application claims the benefit of U.S. Provisional Patent Application No. 63/588,828, filed Oct. 9, 2023, the entire contents of which is incorporated by reference for all purposes as if fully set forth herein.

### FIELD OF DISCLOSURE

The present disclosure relates to a bone screw drill targeting guide system and method that can be used in minimally-invasive bone fusion surgical procedures.

### BACKGROUND

In a minimally-invasive joint fusion procedure, it is a challenge to provide an implant construct with the robustness of a fusion plate while remaining minimally invasive. This is especially true while avoiding periosteal stripping to preserve good blood supply to a healing fusion site.

Conventionally, surgeons can use a screw construct to fuse bones or joints while remaining minimally invasive, but this does not always address the need for a stronger fixation construct, such as a plate, with more points of fixation into a bone. Existing plate constructs typically require substantial tissue stripping to ensure that the plate fits well onto bone.

### SUMMARY

To overcome the problems described above, embodiments of the present disclosure provides a minimally-invasive implant insertion system and method for bone joint fusion including a stability screw trajectory and guiding holes to place screws to secure an implant. A wire, pre-drill, and broaching system can be used to create a pocket for the implant that can be a partially intramedullary style of plate. The implant insertion system and method can be used with minimal tissue stripping.

According to an embodiment, a system for bone fusion includes an implant configured to be attached to two bone portions; an insertion handle attached to the implant by a threaded drill guide and including a stability screw trajectory guide portion and an implant screw guide portion; and a stability screw configured to pass through the stability screw trajectory guide portion and into the two bones.

The system can further include a pilot hole wire guide that includes an alignment feature to fit the two bone portions and configured to align a trajectory to create a cortical window in a cortical bone of one of the two bone portions.

The system can further include a broach having a rectangular cross-section.

In an aspect, the pilot hole wire guide further includes a broach.

In an aspect, the broach includes a rectangular cross section.

In an aspect, the implant is defined as a plate including an elongated portion and a plurality of screw holes.

In an aspect, a portion of the implant is configured to fit through the cortical window.

In an aspect, a portion of the implant is configured to be attached to an outside of one of the two bone portions.

In an aspect, the threaded drill guide is attached to one of the screw holes.

In an aspect, the insertion handle is radiolucent.

In an embodiment, a method of fusing two bone portions includes creating a cortical window in a bone of the bone joint; inserting an implant into the cortical window using an insertion handle that includes a threaded drill guide attached to the implant; inserting a stability screw in bone of the two bone portions, the trajectory of which is defined by a stability trajectory portion of the insertion handle; and inserting implant screws through cortical bone of the two bone portions and into the implant, the trajectories of which are defined by screw guide portions of the insertion handle.

In an aspect, the creating the cortical window includes drilling a pilot hole and forcing a broach through the pilot hole.

In an aspect, the broach includes a rectangular cross section.

In an aspect, the implant is defined as a plate including an elongated portion and a plurality of screw holes.

The method can further include drilling a pilot hole for the stability screw using a pilot hole wire guide that includes an alignment feature to fit over a portion of the two bone portions.

In an aspect, pilot hole wire guide includes a broach for creating the cortical window.

In an aspect, the alignment feature is patient specific.

In an embodiment, an insertion handle includes a drill guide portion including a first through hole configured to accept a threaded end of a drill passed through the insertion handle and screwed into a threaded hole of a bone implant to secure the bone implant to the insertion handle; and a stability trajectory portion including a second through hole configured to accept a stability screw to fuse a bone portion while the bone implant is in a bone.

In an aspect, the insertion handle further includes a proximal screw portion including a third through hole and configured to accept a drill sleeve to drill a hole in the bone along a trajectory set by the third through hole.

In an aspect, the trajectory is aligned so that a proximal screw can be fed through the screw sleeve, into the hole in the bone, and through a bore of the bone implant.

The above and other features, elements, characteristics, steps, and advantages of the present invention will become more apparent from the following detailed description of preferred embodiments of the present invention with reference to the attached drawings.

### BRIEF DESCRIPTION OF THE DRAWINGS

The inventive concepts of the present disclosure will be described in more detail in conjunction with the following drawing figures. The structures in the drawing figures are illustrated schematically and are not intended to show actual dimensions.

FIG. 1 shows an incision at a bone joint.

FIG. 2 shows a cortical window at the bone joint.

FIG. 3 shows a pilot hole wire guide to align a wire for pre-drilling.

FIG. 4 shows a broach into a pilot hole to create a cortical window.

FIG. 5 shows an inserter handle used to locate an implant.

FIG. 6 shows an implant.

FIG. 7 shows a wire sleeve and adapter for placement of a guide wire for a stability screw.

FIG. 8 shows placement of a stability screw.

FIG. 9 shows assembly of nested drill and screw sleeves for proximal implant screws.

FIG. 10 shows placement of proximal implant screws.

FIG. 11 shows removal of threaded drill guide and placement of distal implant screws.

FIG. 12A to FIG. 12C show the result of the implant.

### DETAILED DESCRIPTION

This description of the exemplary embodiments is intended to be read in connection with the accompanying drawings, which are to be considered part of the entire written description. The drawing figures are not necessarily to scale and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. When only a single machine is illustrated, the term “machine” shall also be taken to include any collection of machines that individually or jointly execute a set (or multiple sets) of instructions to perform any one or more of the methodologies discussed herein. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

Unless otherwise expressly stated, it is in no way intended that any method set forth herein be construed as requiring that its steps be performed in a specific order, nor that with any apparatus, specific orientations be required, unless specified as such. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

Disclosed is a system for and a method of minimally-invasive bone fusion. Fusion can be to two portions of the same bone or to a bone joint with different bones. The bone joint can be any joint in which the geometry of the disclosed system and method will work. As an example, the disclosure is described with respect to fusion of a metatarsal phalangeal (MTP) joint. Figures of this disclosure include bones, and it should be understood that a patient's skin is covering the bones, but is not shown for clarity.

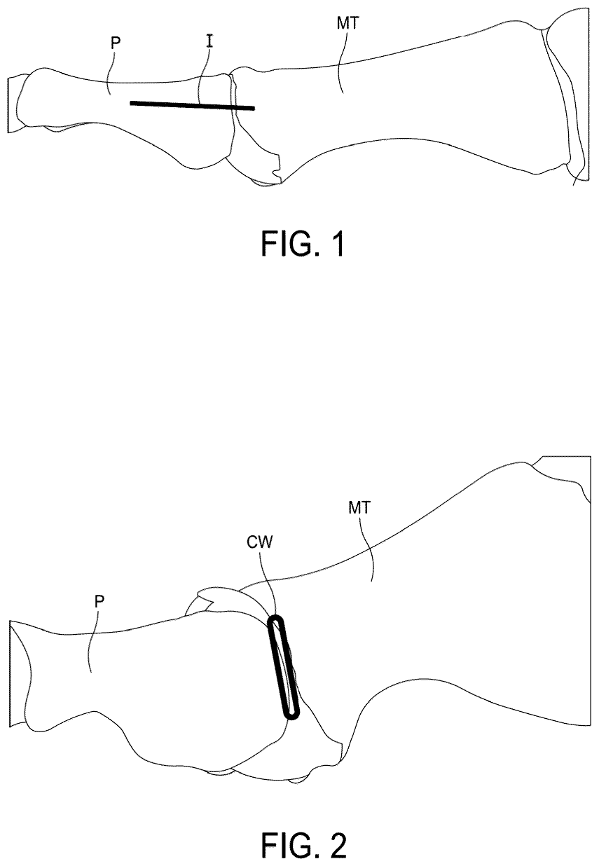

In a system and method of minimally-invasive joint fusion, FIG. 1 shows that during surgery an incision I can be made in the skin (not shown) at the bone area to be fused, as shown the MTP joint of a metatarsal MT and a phalanx P bones. In this procedure, for example, the incision I can extend about 20-30 mm distally, starting at about 5 mm proximal to the joint edge on the medial side.

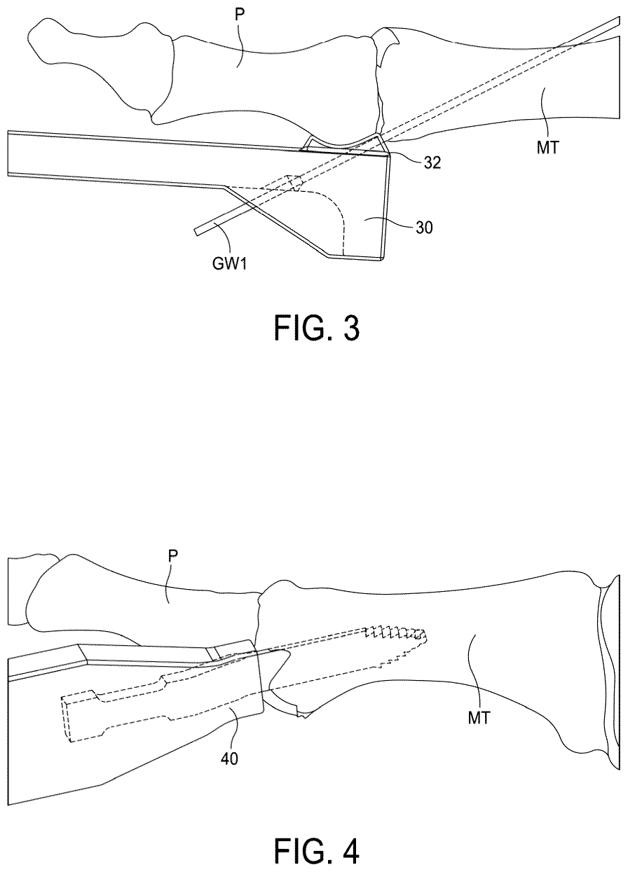

FIG. 2 shows that a substantially rectangular or oval cortical window CW can be made alongside a point where the distal bone (i.e., the proximal phalanx P) touches the proximal bone (i.e., the metatarsal MT). The cortical window CW can be made manually with a burr or another suitable instrument. Additionally, the burr can be used to create a space or pocket inside the bone. Alternatively, the cortical window CW can be made with a wire to guide pre-drilling and broach shown with respect to FIGS. 3 and 4.

FIG. 3 shows that a pilot hole wire guide 30 can be used to assist in aligning tools that create the cortical window CW. Geometry of the pilot hole wire guide 30 can be sized and shaped for the particular joint/bones being fused. For example, the pilot hole wire guide 30 can include an alignment feature such as a concave depression 32 to fit over a portion of a bone to align a wire trajectory. FIG. 3 shows that the concave depression 32 can be fit to a medial flare of a proximal phalanx face to align the trajectory for a guide wire GW1 for placement into the metatarsal MT as close to the MTP joint as possible. The pilot hole wire guide 30 can be located, the guide wire GW1 can be put into place, and a pilot hole drilled (e.g., 3 mm diameter) into the metatarsal MT following the trajectory of the guide wire GW1. The drill can be cannulated so the guide wire GW1 can remain in place during pre-drilling and removed after the pilot hole has been created.

Once a pilot hole is created in a bone, a broach can be forced into the pilot hole to create the cortical window CW. FIG. 4 shows a rectangularly-shaped broach 40 that can be used to create the cortical window in the metatarsal MT. For example, the broach 40 can be defined in an end of the pilot hole wire guide 30. After the pilot hole is drilled, the pilot hole wire guide 30 can be flipped around and the broach at the other end from the depression 32 located in the pilot hole and impacted through the cortical bone and pulled out to define the rectangular cortical window CW. The broach 40 can include a rectangular cross section to displace cancellous bone to effectively create a rectangular pocket. The broach 40 can be fluted with cutting teeth as shown, or be smooth without cutting teeth.

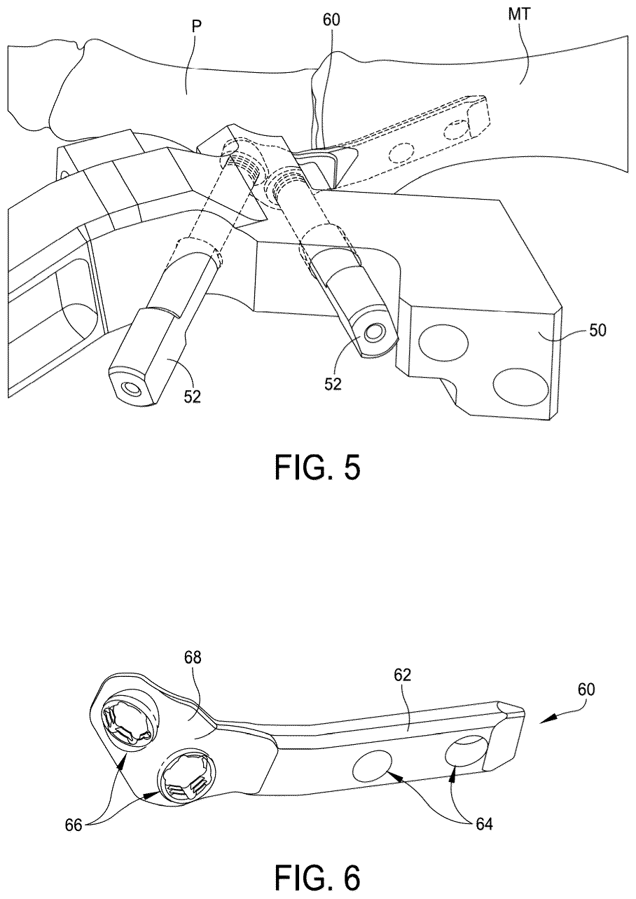

As described, the rectangular pocket can be created manually with a burr, or via the broaching system described above. Once the cortical window CW and the cancellous pocket has been made, an implant 60 can be inserted through the cortical window CW into the bone, as shown in FIG. 5. For example, the implant 60 can be defined as shown in FIG. 6 to include an elongated portion 62 with a rectangular cross section, two bores 64 through the elongated portion 62, and two threaded holes 66 through a curved portion 68. Alternatively, the two bores 64 can be replaced by threaded holes. The geometry of the implant 60 can be defined for the joint being fused and be patient specific to fit a patient's unique anatomy. The implant 60 can be made of a titanium alloy or any other suitable material.

Referring to FIG. 5, the implant 60 can be inserted into the cortical window CW and positioned in the bone with the aid of an insertion handle 50 and threaded drill guides 52. The drill guides 52 can each include a threaded end that is passed through the insertion handle 50 and screwed into the threaded holes 66 of the implant 60 to secure the implant to the insertion handle 50. The surgeon can use the insertion handle 50 to orient and insert the implant 60 and provide alignment guiding for the stability screws and the implant screws as further described. The insertion handle 50 and the threaded drill guides 52 can be radiolucent.

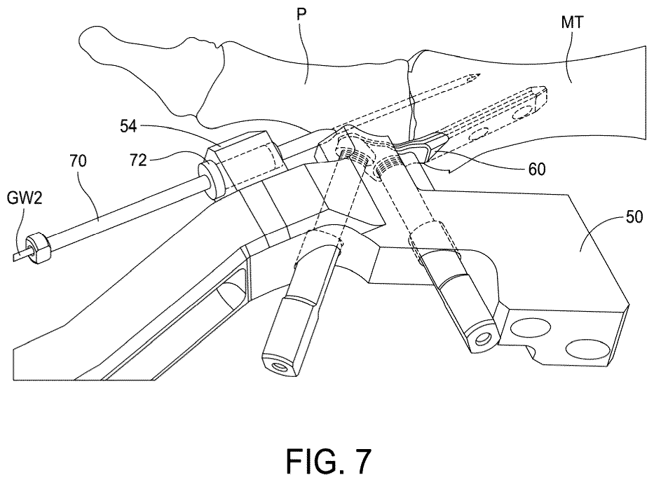

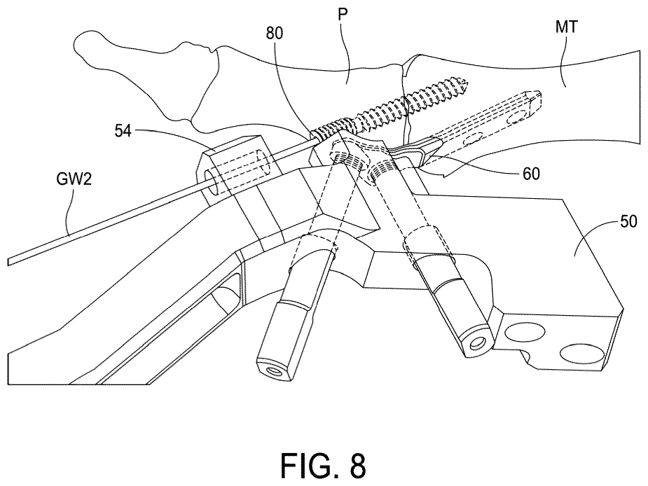

FIG. 7 shows that a wire sleeve 70 and a wire sleeve adapter 72 can be placed into a stability trajectory portion 54 of the insertion handle 50. The stability trajectory portion 54 is arranged to align a hole through the stability trajectory portion 54 toward the joint or bone portions to be fused for insertion of a stability screw 80, shown in FIG. 8. Although shown as two components, the wire sleeve 70 and the wire sleeve adapter 72 can be combined as one piece. Once the wire sleeve 70 and the wire sleeve adapter 72 are in place, a K-Wire GW2 (e.g., 1.4 mm diameter) can be used to create the trajectory for the stability screw 80. The stability screw 80 stabilizes portions of the patient's anatomy and does not interface with the implant 60. Once the K-wire GW2 is in place, the wire sleeve 70 and the wire sleeve adapter 72 can be removed, the depth gauged, and a hole in the joint for the stability screw 80 pre-drilled. A diameter of the hole through the stability trajectory portion 54 can be predetermined to be compatible with the drill and the stability screw 80. The stability screw 80 can be threaded over the K-wire GW2 and be a fully threaded, headless screw to maximize locking into all available cortices and provide suitable stability of the bones at the joint or bone portions to be fused. For example, the stability screw 80 can be a 4 mm MICA™ Screw. The K-wire GW2 can be removed once the stability screw 80 is in place.

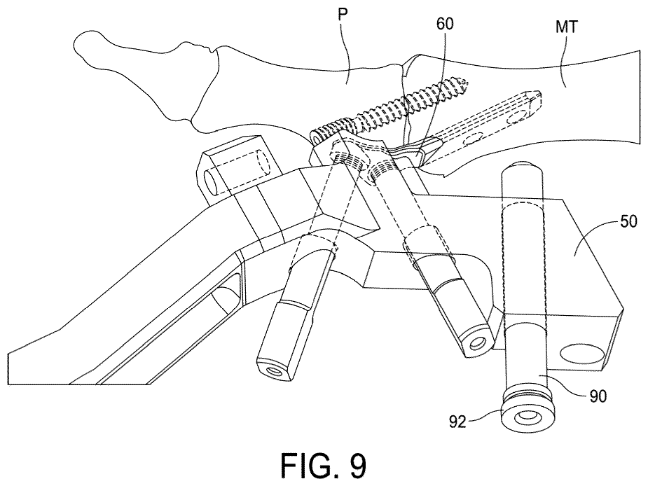

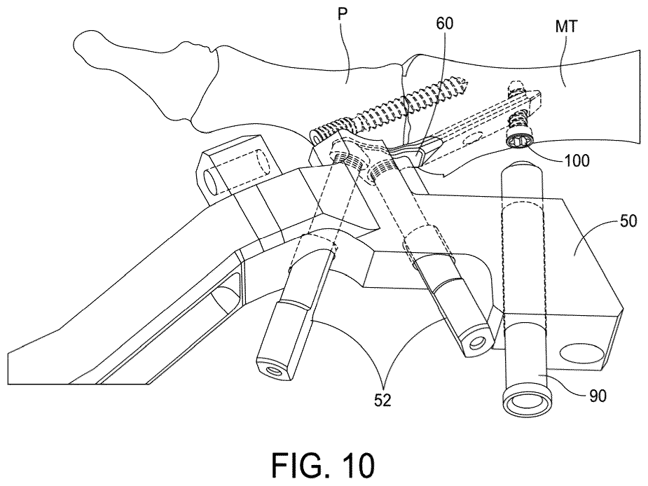

FIGS. 9 and 10 are used to show how proximal screws 100 can be placed to secure the implant 60. This can be done via a nested screw and drill sleeve. As shown in FIG. 9, a drill sleeve 92 can be nested in a screw sleeve 90. The drill sleeve 92 is sized to accept an appropriately sized pre-drill to drill holes in the bone (shown as the metatarsal MT) along trajectories set by holes through the insertion handle 50. Depth gauging can occur after pre-drilling but before screw placement. After pre-drilling, the drill sleeve 92 can be removed. The screw sleeve 90 is sized to accept the proximal screws 100 that are fed through the screw sleeve 90, along the predetermined trajectory and through the bores 64 of the implant 60. The precise design of the proximal screws 100 can vary. As shown, a screw with a thread on the screw head is used for extra locking into the medial bone cortex, but a screw with a low-profile non-threaded head can also be used.

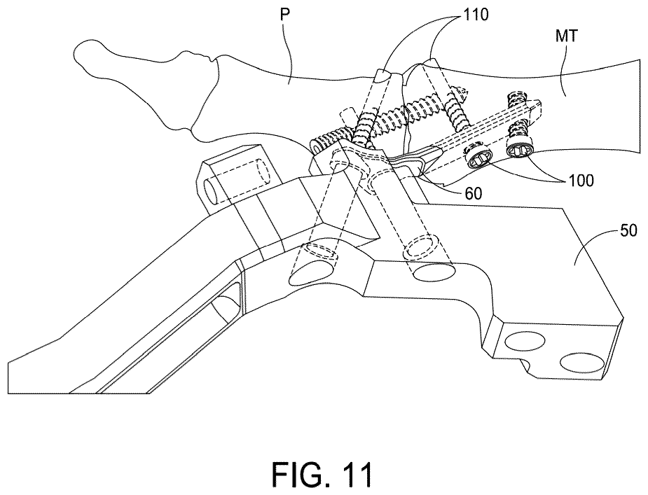

The distal screws 110 for the implant 60, shown in FIG. 11, can be can be placed to secure the implant 60 in a similar manner to the proximal screws 100. The threaded drill guides 52 can be used to pre-drill holes in the bone for the distal screws 110. After the holes have been drilled, the threaded drill guides 52 can be removed one at a time and the distal screws 110 can be inserted after depth gauging. The distal screws 110 are threaded to mate with the threaded holes 66 of the implant 60. For example, the distal screws 110 can be locking plate screws. FIG. 11 shows the orientation of the insertion handle 50 with respect to the distal screws 110 and the implant 60, but would not be in place once the threaded drill guides 52 have been removed.

The specific angle of the plate screws 100, 110 can vary. In some instances, the stability screw 80 can be placed dorsally above the plate screws 100, 110 and in some instances, the stability screw 80 can be placed below the plate screws 100, 110. In either case, trajectories for the plate screws 100, 110 can be adjusted accordingly to avoid interference with the stability screw 80.

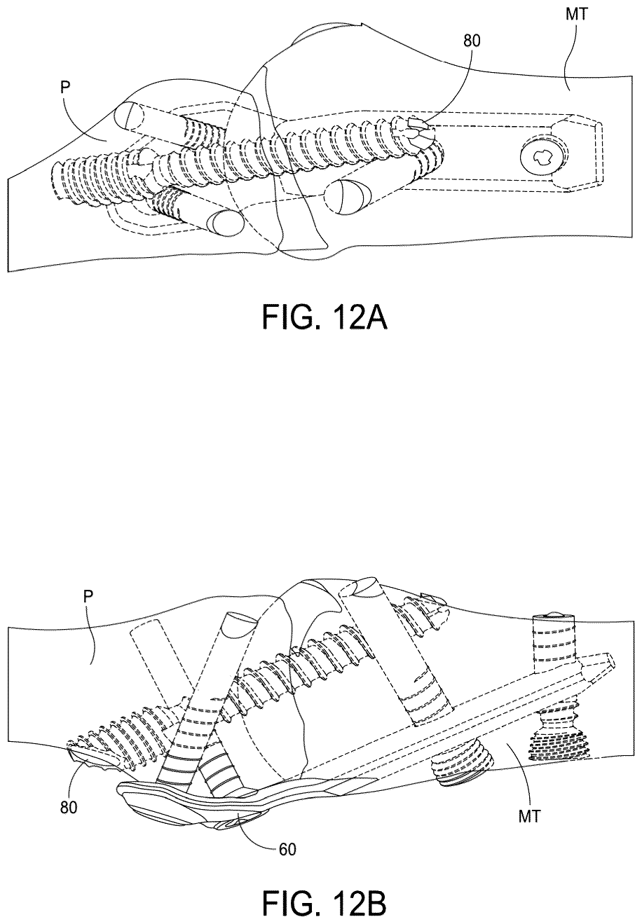

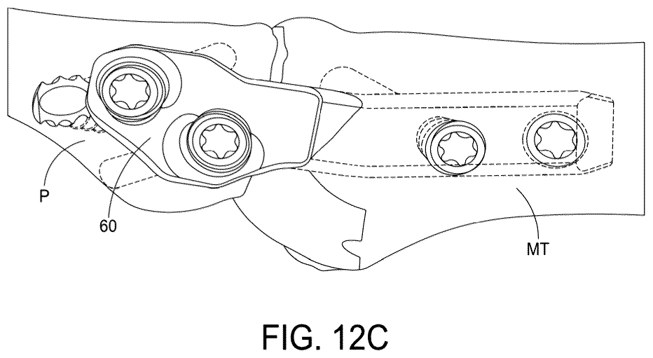

FIGS. 12A-12C are different views of a result of a completed MIS joint fusion procedure that includes a 4-hole plate implant 60 with two plate screws on each side of the MTP joint, along with a stability screw outside the implant 60, all in place while tissue stripping was minimized.

It should be understood that the foregoing description is only illustrative of the present invention. Various alternatives and modifications can be devised by those skilled in the art without departing from the present invention. Accordingly, the present invention is intended to embrace all such alternatives, modifications, and variances that fall within the scope of the appended claims.

What is claimed is:

1. A system for bone fusion comprising:
an implant configured to be attached to two bone portions;
an insertion handle attached to the implant by a threaded drill guide and including a stability screw trajectory guide portion and an implant screw guide portion; and
a stability screw configured to pass through the stability screw trajectory guide portion and into the two bones.

2. The system of claim 1, further comprising a pilot hole wire guide that includes an alignment feature to fit the two bone portions and configured to align a trajectory to create a cortical window in a cortical bone of one of the two bone portions.

3. The system of claim 1, further comprising a broach having a rectangular cross-section.

4. The system of claim 2, wherein the pilot hole wire guide further includes a broach.

5. The system of claim 4, wherein the broach includes a rectangular cross section.

6. The system of claim 1, wherein the implant is defined as a plate including an elongated portion and a plurality of screw holes.

7. The system of claim 2, wherein a portion of the implant is configured to fit through the cortical window.

8. The system of claim 7, wherein a portion of the implant is configured to be attached to an outside of one of the two bone portions.

9. The system of claim 6, wherein the threaded drill guide is attached to one of the screw holes.

10. The system of claim 1, wherein the insertion handle is radiolucent.

11. A method of fusing two bone portions comprising:
creating a cortical window in a bone of the two bone portions;
inserting an implant into the cortical window using an insertion handle that includes a threaded drill guide attached to the implant;
inserting a stability screw in a bone of the two bone portions, the trajectory of which is defined by a stability trajectory portion of the insertion handle; and
inserting implant screws through cortical bone of the two bone portions and into the implant, the trajectories of which are defined by screw guide portions of the insertion handle.

12. The method of claim 11, wherein the creating the cortical window includes drilling a pilot hole and forcing a broach through the pilot hole.

13. The method of claim 12, wherein the broach includes a rectangular cross section.

14. The method of claim 11, wherein the implant is defined as a plate including an elongated portion and a plurality of screw holes.

15. The method of claim 11, wherein the insertion handle is radiolucent.

16. The method of claim 11, further comprising drilling a pilot hole for the stability screw using a pilot hole wire guide that includes an alignment feature to fit over a portion of the two bone portions.

17. The method of claim 16, wherein pilot hole wire guide includes a broach for creating the cortical window.

18. The method of claim 16, wherein the alignment feature is patient specific.

19. An insertion handle comprising:
a drill guide portion including a first through hole configured to accept a threaded end of a drill guide passed through the insertion handle and screwed into a threaded hole of a bone implant to secure the bone implant to the insertion handle; and
a stability trajectory portion including a second through hole configured to accept a stability screw to fuse a bone portion while the bone implant is in a bone.

20. The insertion handle of claim 19, further comprising a proximal screw portion including a third through hole and configured to accept a drill sleeve to drill a hole in the bone along a trajectory set by the third through hole.

21. The insertion handle of claim 20, wherein the trajectory is aligned so that a proximal screw can be fed through the screw sleeve, into the hole in the bone, and through a bore of the bone implant.
